(19) United States
(12) Patent Application Publication
(10) Pub. No.: US 2026/0232210 A1
(43) Pub. Date: Aug. 13, 2026

(54) METHOD AND DEVICE FOR CALCULATING BLOOD PRESSURE LEVEL USING FEATURE VALUE OF PHOTOPLETHYSMOGRAM

(71) Applicants: Youn Jun KIM, Seoul (KR); BIOCONNECT INC, Seoul (KR)

(72) Inventors: Youn Jun KIM, Seoul (KR); Young Su JEON, Gwacheon-si (KR); Jae Hyun LEE, Cheonan-si (KR)

(21) Appl. No.: 18/879,318

(22) PCT Filed: Dec. 11, 2023
(86) PCT No.: PCT/KR2023/020311

§ 371 Date: May 5, 2026

(30) Foreign Application Priority Data
Dec. 13, 2022 (KR) 10-2022-0173885

(51) Int. Cl. A61B 5/024; A61B 5/00

(52) U.S. Cl. CPC A61B 5/02416; A61B 5/7239; A61B 5/7264

(57) ABSTRACT

The present disclosure relates to a method and a device for calculating a blood pressure level using a feature value of a photoplethysmogram. The method, according to one embodiment of the present disclosure, may comprise: receiving photoplethysmogram data including a photoplethysmogram waveform; standardizing the photoplethysmogram waveform included in the photoplethysmogram data; by using the standardized photoplethysmogram waveform, detecting a feature value for calculating a blood pressure level; and calculating the blood pressure level on the basis of the photoplethysmogram data and the detected feature value.

### TECHNICAL FIELD

The present invention relates to a method and a device for calculating a blood pressure level using a feature value of a photoplethysmogram.

### BACKGROUND ART

Whenever the heart contracts, blood is supplied from the heart to the whole body through the aorta, and in this case, a pressure of the aorta changes. This pressure change is transmitted to the peripheral small arteries of the hands and feet, and a photoplethysmogram is a waveform that expresses the volume change of the peripheral blood vessel according to the change in internal pressure of the arteries.

The volume of the blood vessel is changed by the pulsation, and when light having a predetermined wavelength such as infrared rays or visible rays is provided to the blood vessel, the amount of light absorbed varies as the volume of the blood vessel increases or decreases. For example, when 100% of light is emitted, the amount of light that is not absorbed but reflected may change as the pulse increases.

Using this principle, after the light is emitted through a light emitting unit, the speed or amount of reflected infrared rays may be input to a light receiving unit, and the photoplethysmogram may be measured using the feature that the current and voltage are different depending on the speed or amount of the input infrared rays.

However, since the result of the photoplethysmogram may vary depending on the physical condition of a subject, the environmental condition at the time of measurement, and the like, there is a problem that an inaccurate result may be derived when blood pressure is measured using the photoplethysmogram.

The above-described background art is technical information possessed by the inventor for the derivation of the present invention or acquired in the derivation process of the present invention, and may not be necessarily regarded as a known technology disclosed to the general public before the application of the present invention.

### Disclosure

### Technical Problem

The present invention is to provide a method and a device for calculating a blood pressure level using a feature value of a photoplethysmogram. In addition, the present invention is to provide a computer-readable recording medium recorded with a program for executing the method by a computer.

The problems to be solved by the present invention are not limited to the problems described above, and other problems and advantages of the present invention that are not described may be understood by the following description, and will be more clearly understood by the embodiments of the present invention. In addition, it will be appreciated that the problems and advantages to be solved by the present invention may be realized by means and a combination thereof described in the claims.

### Technical Solution

As a technical solution for achieving the above-described technical feature, a first aspect of the present disclosure may provide a method for calculating a blood pressure level using a feature value of a photoplethysmogram, in which the method includes: receiving photoplethysmogram data including a photoplethysmogram waveform; standardizing the photoplethysmogram waveform included in the photoplethysmogram data; detecting a feature value for calculating the blood pressure level by using the standardized photoplethysmogram waveform; and calculating the blood pressure level based on the photoplethysmogram data and the detected feature value.

A second aspect of the present disclosure may provide a computing device, which is a device for calculating a blood pressure level using a photoplethysmogram, in which the device includes: at least one memory; and at least one processor, wherein the at least one processor receives photoplethysmogram data including a photoplethysmogram waveform, standardizes the photoplethysmogram waveform included in the photoplethysmogram data, detects a feature value for calculating the blood pressure level by using the standardized photoplethysmogram waveform, and calculates the blood pressure level based on the photoplethysmogram data and the detected feature value.

A third aspect of the present disclosure may provide a computer-readable recording medium recorded with a program for executing the method of the first aspect by a computer.

In addition, other methods, other systems, and computer-readable recording media storing a computer program for executing the methods in order to implement the present invention may be further provided.

Other aspects, features, and advantages other than those described above will become apparent from the following drawings, the claims, and the detailed description of the present invention.

### Advantageous Effects

According to the technical solution of the present disclosure, the present disclosure may receive the photoplethysmogram data including the photoplethysmogram waveform, standardize the photoplethysmogram waveform, detect the feature value by using the standardized photoplethysmogram waveform, and calculate the blood pressure level based on the photoplethysmogram data and the detected feature value, so that it is possible to calculate the blood pressure level from the photoplethysmogram waveform with higher accuracy.

The blood pressure level is corrected based on the photoplethysmogram data and the personal information and environmental information about the blood pressure level, so that it is possible to calculate the blood pressure level from the photoplethysmogram waveform with higher accuracy.

The effects of the present invention are not limited to the aforementioned effects, and other effects not described above may be evidently understood by a person having ordinary skill in the art to which the present invention pertains from the following description.

### DESCRIPTION OF DRAWINGS

FIG. 1 is a view for explaining one example of a system for calculating a blood pressure level by using photoplethysmogram data according to one embodiment.

FIG. 2 is a configuration view showing one example of a user terminal according to one embodiment.

FIG. 3 is a flowchart for explaining one example of a method for calculating a blood pressure level using a feature value of a photoplethysmogram according to one embodiment.

FIG. 4 is a view for explaining one example in which a processor detects a feature value using the standardized photoplethysmogram waveform according to one embodiment.

FIG. 5 is a view for explaining one example in which the processor acquires a blood pressure level using a first neural network model according to one embodiment.

FIG. 6 is a view for explaining one example in which the processor corrects the blood pressure level using a correction computation model according to one embodiment.

### BEST MODE FOR INVENTION

The method according to one embodiment of the present disclosure may include: receiving photoplethysmogram data including a photoplethysmogram waveform; standardizing the photoplethysmogram waveform included in the photoplethysmogram data; detecting a feature value for calculating the blood pressure level by using the standardized photoplethysmogram waveform; and calculating the blood pressure level based on the photoplethysmogram data and the detected feature value.

### MODE FOR INVENTION

Hereinafter, various embodiments of the present disclosure will be described with reference to the accompanying drawings. Various embodiments of the present invention may be variously modified and have various embodiments, and thus specific embodiments will be illustrated in the drawings and described in detail in the detailed description. However, this is not intended to limit various embodiments of the present disclosure to specific embodiments, and it should be understood that the embodiments include all modifications, equivalents, and substitutes included in the spirit and technical scope of various embodiments of the present disclosure. Regarding the description of the drawings, similar reference numerals are used for similar components.

The expression such as “Include” or “may include” used in various embodiments of the present disclosure indicates the presence of a corresponding function, operation, component, etc., which has been disclosed, and does not limit one or more additional functions, operations, components, etc. In addition, it should be understood that the term such as “include” or “have” in various embodiments of the present disclosure are intended to designate the presence of stated features, numbers, steps, operations, components, parts, or combinations thereof, but do not preclude the presence or addition of one or more other features, numbers, steps, operations, components, parts, or combinations thereof.

In various embodiments of the present disclosure, the expressions such as “or” include any and all combinations of words listed together. For example, “A or B” may include A, may include B, or may include both A and B.

The expressions such as “first”, “second”, “1st”, or “2nd” used in various embodiments of the present disclosure may modify various components of various embodiments, but do not limit the components. For example, the above expressions do not limit the order and/or importance of the components. The above expressions may be used only to distinguish one component from another component. For example, both the first user device and the second user device are user devices, and represent different user devices. For example, a first component may be referred to as a second component and vice versa without departing the scope of various embodiments of the present disclosure.

When a component is referred to as being “connected” or “coupled” to another component, it should be understood that the component may be directly connected or coupled to another component, but a new component may be present between the component and another component. In contrast, when a component is referred to as being “directly connected” or “directly coupled” to another component, it should be understood that a new component may not be present between the component and another component.

In an embodiment of the present disclosure, the terms such as “module”, “unit”, “part”, and the like are terms for referring to a component that performs at least one function or operation, and such a component may be implemented in hardware or software or a combination of hardware and software. In addition, a plurality of “modules”, “units”, “parts”, and the like may be integrated into at least one module or chip and implemented as at least one processor, except for a case where each of the modules, units, parts, and the like needs to be implemented as individual specific hardware.

The terms used in various embodiments of the present disclosure are only for the purpose of describing particular embodiments and are not intended to limit various embodiments of the present disclosure. The singular expression also includes the plural meaning as long as it does not differently mean in the context.

Some embodiments of the present disclosure may be described in terms of functional block components and various processing steps. Some or all of these functional blocks may be implemented with various numbers of hardware and/or software configurations that perform specific functions. For example, the functional blocks of the present disclosure may be implemented by one or more microprocessors or by circuit configurations for a certain function. In addition, for example, the functional blocks of the present disclosure may be implemented in various programming or scripting languages. The functional blocks may be implemented as algorithms executed on one or more processors. In addition, the present disclosure may employ the related art for electronic environment setting, signal processing, and/or data processing. The terms such as “mechanism”, “element”, “means”, and “configuration” may be used widely and are not limited to mechanical and physical configurations.

In addition, connection lines or connection members between components illustrated in the drawings are merely illustrative of functional connections and/or physical or circuit connections. In an actual device, a connection between components may be indicated by various functional connections, physical connections, or circuit connections that are replaceable or added.

Unless otherwise defined, all terms used herein, including technical or scientific terms, have the same meaning as commonly understood by one of ordinary skill in the art to which various embodiments of the present disclosure belong.

Terms such as those defined in commonly used dictionaries should be interpreted as having a meaning consistent with the contextual meaning of the related art and should not be interpreted as either ideal or overly formal in meaning unless explicitly defined in various embodiments of the present disclosure.

Hereinafter, various embodiments of the present invention will be described in more detail with reference to the accompanying drawings.

FIG. 1 is a view for explaining one example of a system for calculating a blood pressure level by using photoplethysmogram data according to one embodiment.

Referring to FIG. 1, a system 1 includes a user terminal 10 and a server 20. For example, the user terminal 10 and the server 20 may be connected by a wired or wireless communication method to transmit and receive data (for example, photoplethysmogram data including a photoplethysmogram waveform) to and from each other.

For convenience of description, FIG. 1 shows that the user terminal 10 and the server 20 are included in the system 1, but the present invention is not limited thereto. For example, the system 1 may include another external device (not shown), and operations of the user terminal 10 and the server 20, which will be described below, may be implemented by a single device (for example, the user terminal 10 or the server 20).

The user terminal 10 may be a computing device equipped with a display device and a device for receiving a user input (for example, a keyboard, a mouse, etc.), and including a memory and a processor. For example, the user terminal 10 may be a notebook PC, a desktop PC, a laptop, a tablet computer, a smartphone, or the like, but the present invention is not limited thereto.

The server 20 may be a device that communicates with an external device (not shown) including the user terminal 10. For example, the server 20 may be a device that stores various data including photoplethysmogram data that includes a photoplethysmogram waveform, and in some cases, may be a device having its own computational capability. For example, the server 20 may be a cloud server, but the present invention is not limited thereto.

The user terminal 10 calculates a blood pressure level using the photoplethysmogram data. In addition, the user terminal 10 corrects the blood pressure level based on personal information and environmental information about the calculated blood pressure level.

The system 1 according to one embodiment calculates the blood pressure level using the photoplethysmogram data. Specifically, the user terminal 10 receives photoplethysmogram data including a photoplethysmogram waveform, standardizes the photoplethysmogram waveform included in the photoplethysmogram data, detects a feature value for calculating the blood pressure level using the standardized photoplethysmogram waveform, and calculates the blood pressure level based on the photoplethysmogram data and the detected feature value. A user 30 may confirm the blood pressure level that is more accurately calculated using the photoplethysmogram data and the feature value.

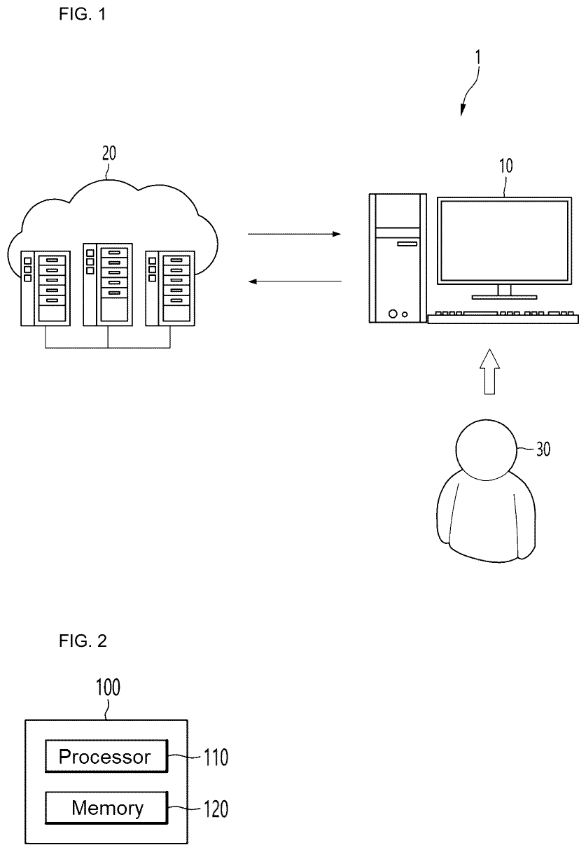

Hereinafter, examples in which the user terminal 10 calculates the blood pressure level using the photoplethysmogram data will be described with reference to FIGS. 2 to 6. Meanwhile, an operation to be described below with reference to FIGS. 2 to 6 may be performed in the server 20 as described above with reference to FIG. 1.

FIG. 2 is a configuration view showing one example of a user terminal according to one embodiment.

Referring to FIG. 2, a user terminal 100 includes a processor 110 and a memory 120. For convenience of description, only components related to the present invention are shown in FIG. 2. In addition to the components shown in FIG. 2, other general-purpose components may be further included in the user terminal 100. For example, the user terminal 100 may include an input/output interface (not shown) and/or a communication module (not shown). In addition, the processor 110 and the memory 120 shown in FIG. 2 may be implemented as independent devices, which is obvious to those skilled in the art related to the present invention.

The processor 110 may process instructions of a computer program by performing basic arithmetic, logic, and input/output operations. The instructions herein may be provided from the memory 120 or an external device (for example, the server 20). In addition, the processor 110 may control overall operations of other components included in the user terminal 100.

The processor 110 calculates the blood pressure level using the photoplethysmogram data. Specifically, the processor 110 receives the photoplethysmogram data including the photoplethysmogram waveform. In addition, the processor 110 standardizes the photoplethysmogram waveform included in the photoplethysmogram data. In addition, the processor 110 detects a feature value for calculating the blood pressure level by using the standardized photoplethysmogram waveform. In addition, the processor 110 calculates the blood pressure level based on the photoplethysmogram data and the detected feature value.

Specific examples in which the processor 110 according to one embodiment operates will be described with reference to FIGS. 2 to 6.

The processor 110 may be implemented as an array of a plurality of logic gates, or may be implemented as a combination of a general-purpose microprocessor and a memory in which a program executable in the microprocessor is stored. For example, the processor 110 may include a general-purpose processor, a central processing unit (CPU), a microprocessor, a digital signal processor (DSP), a controller, a microcontroller, a state machine, and the like. In some environments, the processor 110 may include an application specific integrated circuit (ASIC), a programmable logic device (PLD), a field programmable gate array (FPGA), or the like. For example, the processor 110 may refer to a combination of processing devices such as a combination of a digital signal processor (DSP) and a microprocessor, a combination of a plurality of microprocessors, a combination of one or more microprocessors coupled with a digital signal processor (DSP) core, or a combination of any other such configurations.

The memory 120 may include any non-transitory computer-readable recording medium. As one example, the memory 120 may include a permanent mass storage device such as a random access memory (RAM), a read only memory (ROM), a disk drive, a solid state drive (SSD), a flash memory, and the like. As another example, the permanent mass storage device such as ROM, SSD, flash memory, disk drive, and the like may be a separate permanent storage device that is distinct from the memory. In addition, the memory 120 may store an operating system (OS) and at least one program code (for example, a code for performing an operation to be described below with reference to FIGS. 2 to 6 by the processor 110).

Such software components may be loaded from a computer-readable recording medium separately from the memory 120. Such a separate computer-readable recording medium may be a recording medium that may be directly connected to the user terminal 100, and may include, for example, a computer-readable recording medium such as a floppy drive, a disk, a tape, a DVD/CD-ROM drive, or a memory card. Alternatively, the software components may be loaded into the memory 120 through a communication module (not shown) rather than the computer-readable recording medium. For example, at least one program may be loaded into the memory 120 based on a computer program (for example, a computer program for the processor 110 to perform an operation described below with reference to FIGS. 2 to 6) installed by files provided by developers or a file distribution system for distributing an installation file of an application through the communication module (not shown).

An input/output interface (not shown) may be a unit for an interface with a device (for example, a keyboard, a mouse, etc.) for input or output that may be connected to the user terminal 100 or included in the user terminal 100. The input/output interface (not shown) may be configured separately from the processor 110, but the present invention is not limited thereto, and the input/output interface (not shown) may be included in the processor 110.

The communication module (not shown) may provide a configuration or function for communication between the server 20 and the user terminal 100 through a network. In addition, the communication module (not shown) may provide a configuration or function for communication between the user terminal 100 and another external device. For example, a control signal, a command, data, and the like provided under the control of the processor 110 may be transmitted to the server 20 and/or an external device through the communication module (not shown) and a network.

Meanwhile, although not shown in FIG. 2, the user terminal 100 may further include a display device. Alternatively, the user terminal 100 may be connected to an independent display device in a wired or wireless communication method to transmit and receive data to and from each other.

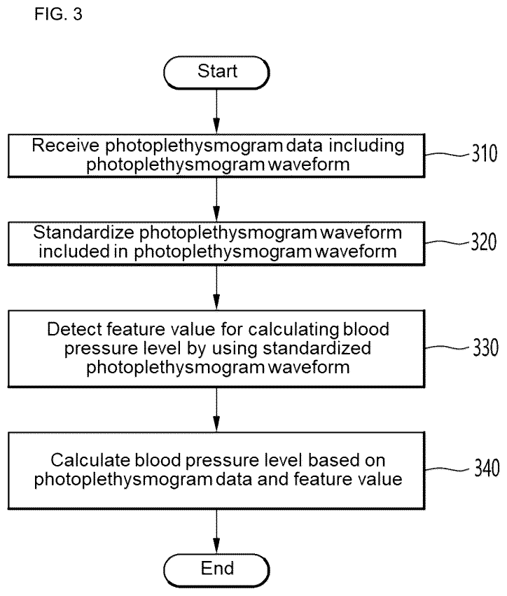

FIG. 3 is a flowchart for explaining one example of a method for calculating a blood pressure level using a feature value of a photoplethysmogram according to one embodiment.

Referring to FIG. 3, the method for calculating a blood pressure level using a feature value of a photoplethysmogram includes steps that are processed in time series by the user terminals 10 and 100 or the processor 110 shown in FIGS. 1 and 2. Therefore, even if omitted below, the contents described above with respect to the user terminals 10 and 100 or the processor 110 shown in FIGS. 1 and 2 may also be applied to the method for calculating a blood pressure level using a feature value of a photoplethysmogram of FIG. 3.

In step S310, the processor receives photoplethysmogram data including a photoplethysmogram waveform.

The photoplethysmogram data may be data including a photoplethysmogram (PPG)-based color difference signal detected from a face region of a user. In addition, the photoplethysmogram data may be data including one of a remote photoplethysmogram (rPPG) waveform and a contactless photoplethysmogram (cPPG) waveform. In addition, the photoplethysmogram data may be data including pulse waves measured from the user through a PPG measuring device, but the present invention is not limited thereto.

In step S320, the processor standardizes the photoplethysmogram waveform included in the photoplethysmogram data.

First, the processor calculates an average value and a standard deviation that correspond to the photoplethysmogram waveform. The processor calculates the average value and the standard deviation from the photoplethysmogram data using the photoplethysmogram waveform corresponding to three periods.

In addition, the processor standardizes the photoplethysmogram waveform using the average value and the standard deviation.

As an example, the processor may standardize the photoplethysmogram waveform using Equation 1 below. The photoplethysmogram waveform included in the photoplethysmogram data may be [x1, x2, x3, . . . , xn] and may be composed of n number of numeric data.

$$z ( x ) = \frac{x - m}{σ} \qquad [ Equation 1 ]$$

Referring to Equation 1, the processor may acquire a standardized photoplethysmogram waveform data value z(x) by using a photoplethysmogram waveform data value x, an average value m, and a standard deviation σ.

In step S330, the processor detects a feature value for calculating the blood pressure level by using the standardized photoplethysmogram waveform.

The processor detects a first feature value corresponding to an amplitude associated with a systolic phase, a second feature value corresponding to an amplitude associated with a diastolic phase, and a third feature value corresponding to a time interval of a waveform reflected from a blood vessel wall, by using the standardized photoplethysmogram waveform.

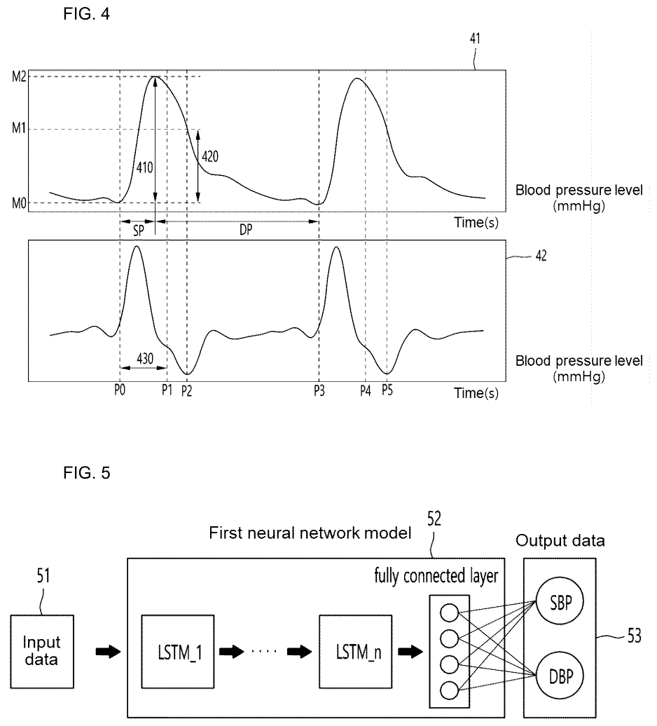

Hereinafter, one example in which the processor detects the feature value using the standardized photoplethysmogram waveform will be described with reference to FIG. 4.

FIG. 4 is a view for explaining one example in which a processor detects a feature value using the standardized photoplethysmogram waveform according to one embodiment. In FIG. 4, a systolic phase SP refers to an interval from a start time P0 of a corresponding period to a time having a maximum blood pressure level M2 of the corresponding period. In FIG. 4, a diastolic phase DP refers to an interval from a time having the maximum blood pressure level M2 of the corresponding period to a time at an end point of the corresponding period.

The processor detects a first feature value 410 corresponding to an amplitude associated with the systolic phase SP, a second feature value 420 corresponding to an amplitude associated with the diastolic phase DP, and a third feature value 430 corresponding to a time interval of a waveform reflected from a blood vessel wall, by using the standardized photoplethysmogram waveform.

The processor detects the first feature value 410 corresponding to the amplitude related to the systolic phase SP by using the maximum blood pressure level M2 corresponding to a peak of a photoplethysmogram waveform 41 and a minimum blood pressure level M0. The first feature value 410 corresponds to an absolute value of a difference between the maximum blood pressure numerical value M2 of the photoplethysmogram waveform 41 and the minimum blood pressure numerical value M0 of the photoplethysmogram waveform 41. On the other hand, the first feature value 410 may be an average value corresponding to all periods included in the photoplethysmogram waveform.

In addition, when a time corresponding to an inflection point having a minimum value in the photoplethysmogram waveform 42 to which differentiation is applied is defined as P2, the processor detects the second feature value 420 corresponding to the amplitude related to the diastolic state DP by using the amplitude of the photoplethysmogram waveform 41 at P2. The second feature value 420 corresponds to an absolute value of the amplitude of the photoplethysmogram waveform 41 at P2. On the other hand, the second feature value 420 may be an average value corresponding to all periods included in the photoplethysmogram waveform.

In addition, when a time corresponding to a point at which an absolute value of a slope decreases and increases after the maximum value in the photoplethysmogram waveform 42 to which differentiation is applied is defined as P1, the processor detects the third feature value 430 corresponding to a time interval of a waveform reflected from the blood vessel wall by using a time from the start time P0 of the corresponding period to P1 in the photoplethysmogram waveform 41. The third feature value 430 corresponds to an absolute value of a difference between the start time P0 of the corresponding period and the time P1 in the photoplethysmogram waveform 41. On the other hand, the third feature value 430 may be an average value corresponding to all periods included in the photoplethysmogram waveform.

Referring back to FIG. 3, in step S340, the processor calculates a blood pressure level based on the photoplethysmogram data and the detected feature value.

The processor calculates a systolic phase blood pressure level and a diastolic phase blood pressure level based on the detected feature value.

Meanwhile, the processor calculates the blood pressure level based on the photoplethysmogram data, the first feature value, the second feature value, and the third feature value.

Meanwhile, the processor may calculate the blood pressure level using a first neural network model. Specifically, the processor inputs the photoplethysmogram data and the detected feature value as input data of the first neural network model. In addition, the processor acquires the blood pressure level based on output data of the first neural network model.

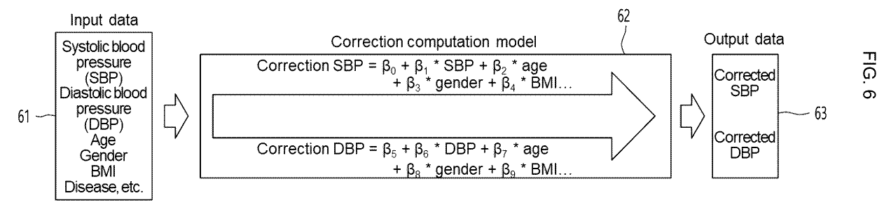

Hereinafter, one example in which the processor acquires the blood pressure level using the first neural network model will be described with reference to FIG. 5.

FIG. 5 is a view for explaining one example in which the processor acquires a blood pressure level using a first neural network model according to one embodiment.

A first neural network model 52 may be any type of deep learning model that acquires a blood pressure level. As one example, the first neural network model 52 includes an RNN-based deep learning model that processes time series data. In FIG. 5, the first neural network model 52 is described as a LSTM deep learning model, but the present invention is not limited thereto. The first neural network model 52 includes a plurality of LSTM layers.

The deep learning model may include a neural network for acquiring a blood pressure level. In the present specification, a neural network, a network function, and a neural network may be used in interchangeable meanings. A neural network may be composed of a set of interconnected computational units, which may generally be referred to as nodes. These nodes may be referred to as neurons. The neural network includes at least one or more nodes. The nodes (or neurons) constituting neural networks may be interconnected by one or more links.

The processor inputs the photoplethysmogram data and the detected feature value as input data 51 of the first neural network model 52. As one example, the processor inputs the photoplethysmogram data, the first feature value, the second feature value, and the third feature value as the input data 51 of the first neural network model 52.

The input data 51 is a vector including the photoplethysmogram data, the first feature value, the second feature value, and the third feature value. As one example, the input data 51 may be a vector including 878 values. The 878 values include 875 values corresponding to the photoplethysmogram waveform included in the photoplethysmogram data, the first feature value, the second feature value, and the third feature value.

The first neural network model 52 inputs the input data 51 to a first LSTM layer LSTM_1. Result values derived from the LSTM layer LSTM_1 are sequentially input to the next LSTM layer, input to an n-th LSTM layer LSTM_n, and input to a fully connected layer.

As the input data 51 is input into the first neural network model 52, the first neural network model 52 outputs a systolic blood pressure level SBP and a diastolic blood pressure level DBP as output data.

Meanwhile, the processor corrects the blood pressure level based on personal information about the blood pressure level and environmental information about the blood pressure level. As one example, the processor may correct the calculated blood pressure level using a cohort model.

The personal information about the blood pressure level includes at least one of gender information, age information, physical information, and disease information of a subject related to the blood pressure level. The personal information about the blood pressure level may be acquired from a database that has been already possessed or acquired from a user's input, but the present invention is not limited thereto.

The environmental information about the blood pressure level includes at least one of temperature information and atmospheric pressure information at the time of measuring the photoplethysmogram waveform.

The processor calculates a weight value by analyzing a correlation between the blood pressure level and the personal information about the blood pressure level and the environmental information about the blood pressure level. As an example, the processor analyzes a correlation with the blood pressure level using the gender information among the personal information to calculate a weight value corresponding to the gender information. As another example, the processor may group age groups using the age information among the personal information, and analyze a correlation between the grouping result and the blood pressure level to calculate a weight value corresponding to the age information. As still another example, the processor analyzes a correlation between an BMI index and the blood pressure level using the body information among the personal information to calculate a weight value corresponding to the body information. As still another example, the processor analyzes a correlation between the temperature, the systolic blood pressure level, and the diastolic blood pressure level using the temperature information among the environment information to calculates a weight value corresponding to the temperature information. For example, the correlation may include a relationship in which the systolic blood pressure level increases by 1.3 mmHg and the diastolic blood pressure level increases by 0.6 mmHg in inverse proportion to a decrease in temperature by 1 degree.

Meanwhile, the processor may correct the blood pressure level using a correction computation model 62. Specifically, the processor calculates a weight value corresponding to the personal information and the environmental information. In addition, the processor inputs the blood pressure level, the personal information, the environmental information, and the weight value into the correction computation model 62. In addition, the processor acquires a blood pressure level corrected using the output data 63 from the correction computation model 62.

Hereinafter, one example in which the processor corrects the blood pressure level using the correction computation model will be described with reference to FIG. 6.

FIG. 6 is a view for explaining one example in which the processor corrects the blood pressure level using a correction calculation model according to one embodiment.

The correction computation model 62 may be any type of machine learning model or deep learning model that acquires a blood pressure level.

The processor inputs the calculated blood pressure level, the personal information about the blood pressure level, the environmental information about the blood pressure level, and the calculated weight value into the correction computation model 62 as the input data 61.

As one example, the correction computation model 62 applies the input data 61 to the following Equations 2 and 3.

$$Corrected SBP = {β}_{0} + {β}_{{1}^{S}} SBP + {β}_{{2}^{S}} Age + {β}_{{3}^{S}} Gender + {β}_{{4}^{S}} BMI \qquad [ Equation 2 ]$$

The processor may acquire a corrected systolic blood pressure level (corrected SBP) by using a weight value β₀, a systolic blood pressure level SBP, a weight value β₁ corresponding to the systolic blood pressure level SBP, a value (age) corresponding to age, a weight value β₂ corresponding to age, a value (gender) corresponding to gender, a weight value β₃ corresponding to gender, a value (BMI) corresponding to BMI, and a weight value β₄ corresponding to BMI.

$$Corrected DBP = {β}_{5} + {β}_{{6}^{S}} DBP + {β}_{{7}^{S}} Age + {β}_{{8}^{S}} Gender + {β}_{{9}^{S}} BMI \qquad [ Equation 3 ]$$

The processor may acquire a corrected diastolic blood pressure level (corrected DBP) by using a weight value β₅, a diastolic blood pressure level DBP, a weight value β₆ corresponding to the diastolic blood pressure level DBP, a value (age) corresponding to age, a weight value β₇ corresponding to age, a value (gender) corresponding to gender, a weight value β₈ corresponding to gender, a value (BMI) corresponding to BMI, and a weight value β₉ corresponding to BMI.

As the input data 61 is input into the correction computation model 62, the correction computation model 62 outputs the corrected systolic blood pressure level (corrected SBP) and the corrected diastolic blood pressure level (corrected DBP) as output data.

As described above, the processor calculates the blood pressure level based on the photoplethysmogram data and the detected feature value using the photoplethysmogram data. The user may confirm the blood pressure level calculated from the photoplethysmogram waveform with higher accuracy.

Meanwhile, the above-described method may be written as a program that may be executed on a computer, and may be implemented in a general-purpose digital computer that operates the program using the computer-readable recording medium. In addition, a structure of data used in the above-described method may be recorded on the computer-readable recording medium through various units. The computer-readable recording medium includes a storage medium such as a magnetic storage medium (for example, ROM, RAM, USB, floppy disk, hard disk, etc.) and an optical reading medium (for example, CD-ROM, DVD, etc.).

It will be understood by those of ordinary skill in the art related to the present embodiment that the present invention may be implemented in a modified form without departing from the essential characteristics of the above description. Therefore, the disclosed methods should be considered from a descriptive point of view rather than a restrictive point of view, and the scope of rights should be construed to include all differences that are described in the claims and are within the scope equivalent thereto, rather than the above description.

1. A method for calculating a blood pressure level using a feature value of a photoplethysmogram, the method comprising:
receiving photoplethysmogram data including a photoplethysmogram waveform;
standardizing the photoplethysmogram waveform included in the photoplethysmogram data;
detecting a feature value for calculating the blood pressure level by using the standardized photoplethysmogram waveform; and
calculating the blood pressure level based on the photoplethysmogram data and the detected feature value.

2. The method of claim 1, wherein the standardizing includes:
calculating an average value and a standard deviation that correspond to the photoplethysmogram waveform; and
standardizing the photoplethysmogram waveform using the average value and the standard deviation.

3. The method of claim 1, wherein the detecting includes detecting a first feature value corresponding to an amplitude associated with a systolic phase, a second feature value corresponding to an amplitude associated with a diastolic phase, and a third feature value corresponding to a time interval of a waveform reflected from a blood vessel wall, by using the standardized photoplethysmogram waveform, and
the calculating includes calculating the blood pressure level based on the photoplethysmogram data, the first feature value, the second feature value, and the third feature value.

4. The method of claim 1, wherein the calculating includes:
inputting the photoplethysmogram data and the detected feature value as input data of a first neural network model; and
acquiring the blood pressure level based on output data of the first neural network model.

5. The method of claim 1, wherein the calculating includes calculating a systolic phase blood pressure level and a diastolic phase blood pressure level based on the detected feature value.

6. The method of claim 1, wherein the method further includes correcting the blood pressure level based on personal information about the blood pressure level and environmental information about the blood pressure level.

7. The method of claim 6, wherein the correcting includes:
calculating a weight value corresponding to the personal information and the environmental information; and
inputting the blood pressure level, the personal information, the environmental information, and the weight value to a correction computation model as input data, and acquiring the corrected blood pressure level from the correction computation model based on output data.

8. A computer-readable recording medium recorded with a program for executing the method of claim 1 by a computer.

9. A computing device for calculating a blood pressure level using a feature value of a photoplethysmogram, the computing device comprising:
at least one memory; and
at least one processor,
wherein the at least one processor receives photoplethysmogram data including a photoplethysmogram waveform, standardizes the photoplethysmogram waveform included in the photoplethysmogram data, detects a feature value for calculating the blood pressure level by using the standardized photoplethysmogram waveform, and calculates the blood pressure level based on the photoplethysmogram data and the detected feature value.
